(19) United States
(12) Patent Application Publication
(10) Pub. No.: US 2008/0032330 A1
(43) Pub. Date: Feb. 7, 2008

(54) PROCESS FOR SELF-ASSEMBLY OF STRUCTURES IN A LIQUID

(76) Inventors: Gafur Zainiev, West Bloomfield, MI (US); Inlik Zainiev, West Bloomfield, MI (US); Timur Zainiev, West Bloomfield, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/835,054

(22) Filed: Aug. 7, 2007

Related U.S. Application Data
(60) Provisional application No. 60/836,103, filed on Aug. 7, 2006.
(60) Provisional application No. 60/905,357, filed on Mar. 7, 2007.

(51) Int. Cl. C12P 21/00; C12M 1/00; C12P 19/34

(52) U.S. Cl. 435/68.1; 435/289.1; 435/91.5

(57) ABSTRACT

A process and apparatus for self-assembling a number of elements and determining their sequence is provided. In the field of DNA analysis, an iterative process is disclosed wherein an apparatus with a set of reaction chambers in which a species of recognition element nucleotides are differentially added and subjected to a polymerization reaction allows recognition of which species is next in sequence on a template strand as measured by a detector in a detection area. Fractional concentration stepwise addition of the identified species then determines if an element repeat exists. The position in the sequence is then completed by addition of a saturating amount of building element to complete the polymerization reaction on all structure strands. The process is repeated until the entire structure is complete and the sequence identified.

### CROSS-REFERENCE TO RELATED APPLICATIONS

This application claims the benefit of U.S. Provisional Patent Applications 60/836,103 filed on Aug. 7, 2006; and 60/905,357 filed on Mar. 7, 2007; the entire disclosures of which are hereby incorporated herein by reference.

### FIELD OF THE INVENTION

The present invention relates to the field of self-assembly of a number of elements into a structure. More particularly, the present invention relates to the assembly of nucleotides to form an oligonucleotide structure and sequence determination thereof. Most particularly, this present invention relates to the field of DNA sequencing.

### BACKGROUND OF THE INVENTION

The art of DNA sequencing, long accomplished by a multi-step brute force approach, was radically transformed by the development of new technologies during the human genome project advancing the pace of sequencing a genome from years to months. Completion of the human genome project saw successful innovations in the fields of recombinant protein engineering, fluorescent dyes, capillary electrophoresis, automation, informatics and process management. (Metzger, M. L., Genome Res, 2005; 15:1767-76).

Modern sequence analysis is most commonly directed toward discovery and analysis of sequence variation as it relates to human health and disease. These continue to be large-scale projects that are plagued by technology that is slow in its application and inaccurate in its nature. Further, current technologies available for sequence analysis tend to require large amounts of nucleic acid template and large biological samples. Important parameters which can be addressed by improved technology include increased sequencing speed, increases in sequence read length achievable during a single sequencing run, decreasing in the amount of template required to obtain positive sequence results, decreasing the amount of reagent required for processing a sequence reaction, improving the accuracy and reliability of the sequences generated, and improved identification of nucleic acid repeats in the strand of DNA.

Several unique approaches are traditionally employed for sequencing DNA. The most common is the dideoxy-termination method of Sanger (Sanger et al., PNAS USA, 1977; 74:563-567). Single nucleotide analysis such as pyro-sequencing first described by Hyman 1988 (Analytical Biochemistry, 174, pages 423-436) has proved to be the most successful non-Sanger method. Cyclic reversible termination or CRT has also been employed with some success. Finally, sequence analysis has been accomplished by an exonuclease reaction wherein particular nucleotide residues are identified in a stepwise fashion as they are removed from the end of an oligonucleotide strand.

The Sanger method represents a mixed mode process coupling synthesis of a complementary DNA template using deoxynucleotides (dNTPs) with synthesis termination by the use of fluorescently labeled dideoxynucleotides (ddNTPs). Balancing reagents between natural dNTPs and ddNTPs leads to the generation of a set of fragments terminating at each nucleotide residue within the sequence. The individual fragments are then detected following capillary electrophoresis so as to resolve the different oligonucleotide strands. The sequence is determined by identification of the fluorescent profile of each length of fragment. This method has proven to be both labor and time intensive and requires extensive pretreatment of the DNA source. Microfluidic devices for the separation of resulting fragments from Sanger sequencing has improved sample injection and even decreased separation times, hence, reducing the overall time and cost of a DNA sequencing reaction. However, the time and labor required to successfully prosecute a Sanger method is still sufficiently great to make several studies beyond the reach of many research labs.

The single nucleotide addition methodology of pyro-sequencing has been the most successful non-Sanger method developed to date. Pyro-sequencing capitalizes on a non-fluorescence technique, which measures the release of inorganic phosphate converted to visible light through a series of enzymatic reactions. This method does not depend on multiple termination events, such as in Sanger sequencing, but instead, relies on low concentration of substrate dNTPs, so as to regulate the rate of dNTP synthesis by DNA polymerase. As such, the DNA polymerase extends from the primer, but pauses when a non-complementary base is encountered until such time as a complementary dNTP is added to the sequencing reaction. This method, over time, creates a pyrogram from light generated by the enzymatic cascade, which is recorded as a series of peaks and corresponds to the order of complementary dNTPs incorporated revealing the sequence of the DNA target. (See Ronaghi, Science, 1998; 281:363-65; Ronaghi, Analytical Biochemistry, 2002; 286:282-288; Langaeet and Ronaghi, Mutational Research, 2005; 573:96-102). While pyro-sequencing has the potential of reducing sequencing time, as well as amount of template required, it is typically limited to identifying 100 bases or less. Further, repeats of greater than five nucleotides are difficult to quantitate using pyro-sequencing methods. Also, pyro-sequencing methods must be carefully designed, as it is the order of dNTP addition that determines the pyrogram profile and investigators must design experiments so as to avoid asynchronistic extensions of heterozygous sequences as almost half of all heterozygous sequences result in asynchronistic extensions at the variable site. (Metzger, 2005).

Cyclic Reversible Termination (CRT) uses reversible terminating deoxynucleotides, which contain a protecting group that serves to terminate DNA synthesis. A termination nucleotide is incorporated, imaged, and then deprotected so that the polymerase reaction may incorporate the next nucleotide in the sequence. CRT has advantages over pyro-sequencing in that all four bases are present during the incorporation phase, not just a single base during a single period of time. Single base addition is achievable through homopolymer repeats and synchronistic extensions are easily maintained past heterozygous bases. Perhaps the greatest advantage of CRT is that it may be performed on many highly parallel platforms, such as high-density oligonucleotide arrays (Pease et al., 1994, and Albert et al., 2003), PTP arrays (Laymon et al., 2003), or random dispersion of single molecules (Nutra and Church, 1999). High-density arrays and incorporation of di-labeled dideoxynucleotide dNTPs by DNA polymerase gives CRT significant improvement in throughput and accuracy. However, CRT suffers several drawbacks including short read lengths that must be overcome before it can be widely employed.

Finally, exonuclease methods sequentially release fluorescently labeled bases as a second step following DNA polymerization to a fully labeled DNA molecule. Using a hydrodynamic flow detector, each dNTP analog is detected by its fluorescent wavelength as it is cleaved by the exonuclease. This method has several drawbacks. For example, the DNA polymerase and, more importantly, the exonuclease must have high activity on the modified DNA strand and generation of a DNA strand fully incorporating four different fluorescent dNTP analogs has yet to be achieved.

Technological advances in fluorescence detection are essential to decrease the amount of target oligonucleotide necessary for sequencing analysis. Four color fluorescent systems such as those employed in Sanger methods have several disadvantages including inefficient excitation of fluorescent dyes, significant spectra overlap between each of the dyes, and inefficient collection of the emission signal. Several dyes have been recently developed that help address these issues, such as fluorescence resonance energy transfer (FRET) dyes (Ju et al., PNAS, 1995; 92:4347-51; Metzger, Science, 1996: 271:1420-1422.) Additional strategies have been proposed, such as fluorescence lifetime and a radio frequency modulation. Finally, Lewis et al. recently described termed pulse multiline excitation (PME) which is an ineffective method for multifluorescence discrimination. (Lewis, PNAS, 2005: 102:5346-41).

The demand for rapid small and large scale DNA sequencing has radically increased over the last several years. Current sequencing methods tend to be expensive and time consuming. Further, the prior art methods each suffer the drawback of inaccuracy in identification of repeat nucleotides in the sequence. Thus, there remains a need for a rapid and accurate sequencing method that can be run on an automated platform.

### SUMMARY OF THE INVENTION

The present process relates to the assembly of structures comprised of a number of elements in a solution wherein the structure is complementary to a target strand. The inventive process is accomplished by providing a set of N recognition chambers. A plurality of templates is added to each chamber. Subsequently, a plurality of recognition elements is added into each chamber with each chamber receiving a homogeneous species of recognition elements that are distinguishable from the recognition elements added to each of the other chambers. The template and recognition elements are then subjected to a polymerization reaction with a plurality of polymerization enzyme so that a complementary recognition element binds and is assembled into the final structure. The process continues by identifying which of the recognition elements is complementary by method of subtraction. Finally, a plurality of building elements, each building element corresponding to the incorporated recognition element, is added to each of N chambers to complete the addition of elements at that step. This sequence is repeated until the structure is complete. By identification of each of the individual elements as it is added to the growing oligomeric structure, the sequence of the template is determined. In a nonlimiting example, if the number of elements in the sequence is 4, N is 4.

Optionally, all of the recognition elements are washed away from each of the recognition chambers prior to addition of building elements. Recognition elements and building elements are optionally selected from the group including nucleotides, ribonucleotides, deoxynucleotides, dideoxynucleotides, peptide nucleotides, modified nucleotides, modified peptide nucleotides, modified phosphate sugar backbone nucleotides, amino acids, or modified amino acids.

In addition to the recognition chambers, the process optionally employs a repeat detection chamber. Repeat detection achieved by small stepwise addition of less than saturated amounts of building or recognition element and detection of free element following each stepwise addition. When the element added is no longer placed in sequence, the particular site on the template is considered saturated and the number of repeat elements in the sequence is calculated.

It is further envisioned that the liquid solution from each of the recognition chambers is optionally transferred to the repeat detection chamber prior to addition of recognition or building elements. After repeat detection, the liquid reaction material is then optionally transferred from the repeat detecting chamber and divided among all N recognition chambers prior to addition of recognition elements. Optionally, all the recognition or building elements are washed out of the repeat detecting chamber or the recognition chambers prior to addition of further elements.

In an alternative embodiment, a sequence construction chamber is additionally employed where solution from the recognition chambers and the repeat detection chamber is transferred to the sequence construction chamber and the structure is increased by addition of complementary building elements. Optionally, a large sequence construction chamber is employed so that after each step of building element addition, a volume of liquid is transferred from the sequence construction chamber back to the repeat detecting chambers, as well as each of the recognition chambers. This template is then added to by new recognition elements to determine what the next element and sequence is.

It is appreciated that the oligonucleotide, or oligomeric template, is immobilized on a support, or free in solution. It is further appreciated that recognition or building elements are optionally washed away and removed from each of the N recognition chambers, the repeat detecting chamber, or the sequence construction chamber so that a clean template can be reutilized upon each subsequent addition, hence regenerating the system. The polymerization enzyme responsible for the polymerization reaction is illustratively a DNA polymerase, an RNA polymerase, a reverse transcriptase, or mixtures thereof.

As opposed to the template being attached to a support, the polymerization enzyme is optionally attached to the support, or is itself free in solution. The nucleic acid polymerizing enzyme is optionally a thermostable polymerase or a thermodegradable polymerase. Template types operable in the instant invention include double-stranded DNA, single-stranded DNA, single-stranded DNA hairpins, RNA, and RNA hairpins. The template is optionally attached to a support by hybridizing to a primer sequence that is itself optionally affixed to a support. The primer sequence is free in solution and is complementary to a small segment of the target sequence so that a polymerization reaction may be extended from the primer.

Recognition elements optionally comprise a label or a plurality of labels. Numerous label types are operable in the instant invention illustratively including chromophores, fluorescent moieties, haptens, enzymes, antigens, dyes, phosphorescent groups, chemiluminescent moieties, scattering or fluorescent nanoparticles, FRET donor or receptor molecules, Raman signal generating moieties, precursors thereof, clevage products thereof; and combinations thereof. In addition, photobleachable, photoquenchable, or otherwise inactivatable labels are similarly operable. The label is optionally attached to a recognition element at any suitable site illustratively including a base, a sugar moiety, an alpha phosphate, beta phosphate, gamma phosphate, or combinations thereof. It is appreciated that each homogeneous species of recognition element optionally carries a label that is distinguishable from other labels on different recognition elements.

Detection of free recognition element is accomplished by one or many of numerous identifying techniques; illustratively, far field microscopy, near field microscopy, evanescent wave or wave guided illumination, nanostructure enhancement, photon excitation, multiphoton excitation, FRET, photo conversion, spectral wavelength discrimination, fluorophore identification, background suppression, mass spectroscopy, chromatography, electrophoresis, surface plasmon resonance, enzyme reaction, fluorescence lifetime measurements, radio frequency modulation, pulsed multiline excitation, or combinations thereof.

Background fluorescence or fluorescence of previously added recognition elements to a growing structure is optionally eliminated by photobleaching the label, cleaving the label, or otherwise inactivating the label. The label is optionally cleaved from the backbone prior or subsequent to addition of recognition or building elements.

The present invention also envisions an apparatus for self-assembly of a number of elements into a structure that comprises a reaction area, a preparation area, which is in fluidic connection with said reaction area, and a detection area, which is in fluidic, physical, or optical connection with the reaction area or preparation area. It is appreciated that the reaction area has no moving parts. Within the reaction area there are N recognition chambers where each chamber has a plurality of microdispensers. Each microdispenser is capable of dispensing a unique species of recognition element or a building element. Optionally, each dispenser is employed to dispense numerous elements simultaneously, or be washed out between the dispensing of a particular element. The chambers within the reaction area are optionally a batch flow reactor, a plug flow reactor, or a drop reactor.

### BRIEF DESCRIPTION OF THE DRAWINGS

The present invention is further detailed with respect to the following nonlimiting figures. These figures depict only particular processes and apparatuses according to the present invention with variants existing beyond those depicted.

FIG. 1A is a schematic of a set of reaction chambers that contain reaction chamber solution 4, template molecule 3, polymerizing enzyme 5, and all other necessary reagents wherein the open site on the template molecule 3 is depicted by a T such that only the chamber with the A recognition element 1 is subjected to a successful polymerization reaction removing the A recognition element from the solution of that chamber alone and allowing detection of free recognition element species 1 in all other chambers leading to identification of which nucleotide species is in the hybridization position;

FIG. 1B1 depicts a primer 6 affixed to a support 7, the primer 6 hybridized to a DNA template molecule wherein a polymerizing enzyme 5 recognizes the 3′ end of the template 3, and the amount of recognition element 1 added to the reaction chamber is less than that required to saturate all hybridization sites on the template strands 3;

FIG. 1B2 depicts alternative schematic immobilization of FIG. 1B1 relative to a support wherein DNA template molecule affixed to a support 7, the DNA template molecule hybridized to a primer 6 wherein a polymerizing enzyme 5 recognizes the 3′ end of the template 3, and the amount of recognition element 1 added to the reaction chamber is less than that required to saturate all hybridization sites on the template strands 3;

FIG. 1C depicts a two reaction chamber protocol wherein a biotin/streptavidin interaction immobilizes a primer 6 to a support 7 and hybridization of the template 3 to the primer immobilizes the template to the support, hence, creating a binding site for a polymerizing enzyme 5 such that a complementary recognition element depicted by a rectangle is able to hybridize with the open site on the template molecule and the polymerizing enzyme binds the complementary recognition element to the growing primer strand to form the structure, whereas the non-homologous recognition element depicted by a triangle in chamber 2 will not be added to the growing structure, furthermore, stepwise addition of the complimentary recognition element species of building element completes the repeat determination step and saturation of all template strands, washing out of all unbound elements allows a repeat of the procedure in the same reaction chambers to complete the assembly and sequence identification of the structure;

FIG. 1D depicts a schematic of chambers 1 and 2 of FIG. 1C wherein an electric potential applied throughout a detection chamber 19 is used to selectively move non-complementary elements from the reaction chamber past a detector to a collection area 18; the collected free recognition elements are optionally returned to the reaction chamber by reversing the polarity of the electric field;

FIG. 2 depicts an alternative embodiment of a reaction chamber 2 wherein recognition elements 1 are flowed over template/primer/polymerase immobilized on a support 7 mediated by a pump 16 and the presence of free recognition element 1 is determined by a detector 9;

FIG. 3 depicts an overall schematic for an apparatus of the instant invention that includes a reaction area 17 that contains reaction chambers 2 each with a plurality of microdispensers 8 that dispense elements into the reaction chamber solution 4, and the reaction chamber is in fluid connection by a fluid communication medium 12 to a repeat detecting chamber 10 and a sequence building chamber 11, the reaction area 17 is in connection with a detection area 14 and a reagent preparation area 13. In a non-limiting example where N is 4, each microdispenser 8 supplies only one homogenous type of mono nucleotide such as A, T, G or C.

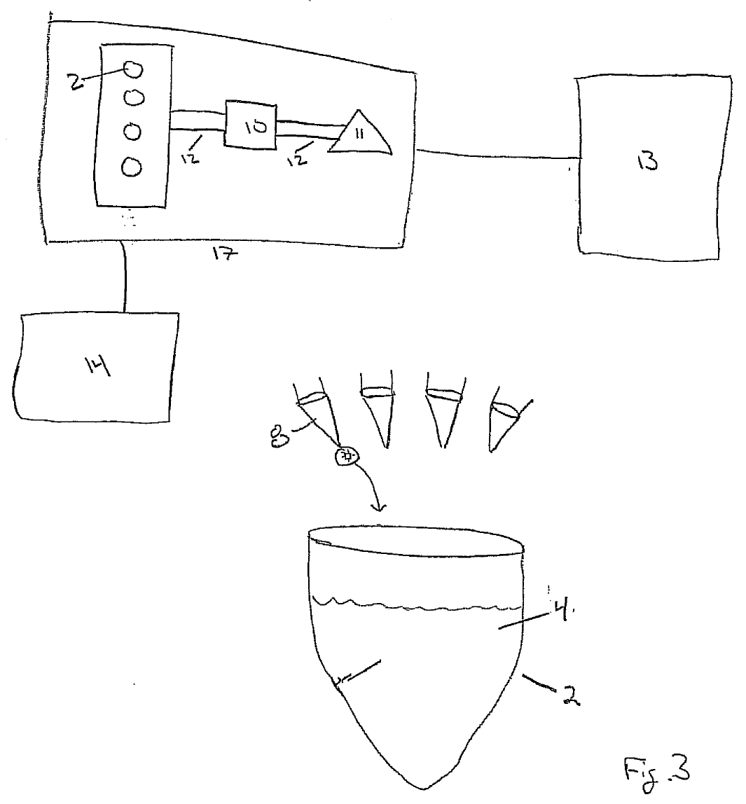

### DETAILED DESCRIPTION OF THE PREFERRED EMBODIMENTS

The present invention relates to a process of identifying individual units in a self-assembling number of elements as they are assembled into a structure. Without limitation, the instant invention is more specifically directed toward sequencing of a deoxyribonucleotide structure into its individual monomer units. Thus, the present invention has utility as a DNA sequencing process and apparatus.

Generally, determining the sequence of a structure containing N different elements requires a system having N sets of chambers. Each of N sets in turn contains N chambers. To initiate the assembly process a different element is placed in each chamber in every set. For example the i-th chamber in every set contains the i-th element. It is appreciated that each element is optionally affixed to a wall or structure or support or to the bottom of a chamber. When the target sequence is unique, i.e. the next element is always different from the previous one, the next step in the process is to put the same element into each chamber of the same set but different elements across sets. That is, all chambers of the i-th set will receive i-th element. Since there are N sets each set containing N chambers, there are N×N possible two element strings. There will be one and only one chamber where a second element will bind to the first element to begin growing the structure. In all other chambers there will be no addition of one element to the other. Thus, by subtraction it is identified what the sequence of the first and second elements are. For example, if the growth of the structure happened in the i-th chamber of the j-th set then the first element in a sequence is the i-th and the second one is the j-th where i and j are integers greater than zero and less than or equal to N.

All sets not containing the chamber wherein a two element structure was formed are discarded such that only one set of N chambers remains. It is appreciated that all chambers are then optionally washed of free unbound elements so that the only remaining structure in the system is the surface bound or solution two element structure. At this point an excess amount of the identified element is added to each chamber of the i-th set so that all chambers contain the same two element structure.

Finding the third and every other element in the sequence is reduced to a simple algorithm. N different elements are added into N chambers to reveal the next element. All chambers are then optionally washed of the elements from all N chambers and the identified element is added to all chambers in excess to grow the structure one more unit. By repeating the steps for every member of the unidentified sequence the element order is easily determined.

In a nonlimiting example, an unidentified DNA template sequence is resolved by the instant inventive process. DNA is comprised of four element types, an adenine, guanine, thiamine, and cytosine. Therefore, the integer N is equal to 4. It is appreciated that DNA is optionally synthesized in vitro in a chamber in the presence of all required molecules illustratively including a DNA polymerase and a helicase. It is further appreciated that with a given sequence of DNA only one of the four types of elements A, T, G or C will be assembled in with the target sequence at each hybridization site being identified. It is known in the art that A hybridizes to T and G hybridizes to C. If the next element in an unknown sequence of DNA is a T only the chamber containing an A recognition element will produce extension, thus, removing the A element from solution. All other chambers will contain free nucleotide. By identifying which wells contain free nucleotide the sequence of the target is deciphered. Thus, according to the inventive process DNA sequencing is illustratively performed using four chambers with the appropriate number of DNA molecule copies in each chamber.

Of primary importance to the inventive process is that each step is conceptually split into many small steps. Each small step consists of supplying one dose of elements wherein the number of DNA template copies is much larger than the number of elements delivered during each small step. Therefore, if a particular element is incorporated into a DNA molecule at the next unoccupied site in the sequence, it is appreciated that the number of free monomers is negligible in solution after the first small step. This process allows simple identification of repeat elements (or copy number of a particular template) in the structure sequence. In a nonlimiting example the total number of DNA copies of template copies in the chamber is ten times larger than the number of elements in each single dose. It is appreciated that the first dose of elements being one-tenth that of the number of DNA will occupy sites on one-tenth of the DNA molecules leaving nine-tenths of the sites on the DNA template molecules free. After ten small additions of elements all sites on the template DNA will be occupied. Between and after each addition one-tenth concentration of element it is appreciated that there will be no free monomer elements in solution because all of the elements will be incorporated into DNA molecules. Upon addition 11 observation of free monomers in the solution occurs which signals completion of the current step and beginning of the next one. As such, a primary advantage of the instant invention over the prior art is a rapid and accurate process for revealing repeat elements or nucleotides in the sequence.

In a nonlimiting example the chamber under consideration contains 100,000 copies of DNA template molecules. One dose of monomer elements contains 10,000 molecules of monomer. Therefore, it will require 10 doses of monomer element to fill the vacancies in all copies of DNA molecules. The eleventh dose will create 10,000 monomer elements available for detection in the solution signaling that the site is not a repeat and, further, signaling the next recognition step.

During sequence identification only one chamber out of four will incorporate elements into the growing DNA structure the other three chambers will demonstrate free monomers in solution, thus, revealing the nature of the monomer which is incorporated into the growing DNA structure. After identification of which monomer element is incorporated at the particular site sufficient copies of that identified monomer element is optionally added to the other three chambers, thus, occupying that site on a growing DNA structure in all four chambers. That completes the final step of the process and determination of the entire sequence of the unknown DNA template molecule is similarly determined.

The elements supplied to the chamber are classified by purpose. The first type of element is a recognizing element. Recognizing elements are elements identifiable as free in solution after being unable to bind in a complementary fashion to the DNA template at the next available site. The second type of element is a building element. Building elements are elements not intended to be used for recognition, however, it is appreciated that they are optionally used as recognizing elements. Building elements are designed to occupy free sites in all growing DNA structures left vacant by the non-complementary recognizing elements in a chamber.

The inventive process is operable for many different types of sequence structures or element containing structures. A recognition element or a building element is illustratively a nucleotide, a ribonucleotide, deoxyribonucleotide, deoxynucleotide, peptide nucleotides, modified nucleotides, modified peptide nucleotides, modified phosphate sugar backbone nucleotides, amino acids, or modified amino acids. Although chemically similar, a recognition element is mainly involved in an inventive process for nucleotide identification or sequencing while a building element is applied for a collateral determination of copy number of a particular DNA template.

Recognition elements are optionally labeled. A single label or multiple labels are optionally present on each individual recognition element. In a nonlimiting example, during DNA sequencing four different types of recognition elements are employed: A, T, G, or C. Each recognition element optionally contains the same label or different labels that are distinguishable from each other based on characteristics of the combination of label and the remainder of the recognition element. Labels are optionally bound to one or multiple sites on a recognition element such as in a nucleotide. Recognition elements optionally have a label attached at a base, on a sugar moiety, on the alpha phosphate, beta phosphate, gamma phosphate, or any combination thereof. Illustratively, a label for adenine preferably has a fluorophore bound to the gamma phosphate wherein the fluorophore is distinguishable from a fluorophore bound to the gamma phosphate on a different species of recognition element. Thus, in the case of DNA sequence the four recognition element species optionally contain four different fluorophores.

Multiple label types are operable in the instant invention illustratively including cliromophores, fluorescent moieties, enzymes, antigens, dyes, phosphorescent groups, chemiluminescent moieties, scattering or fluorescent nanoparticles, Raman signal generating moieties, fluorescence resonance energy transfer donor or acceptor molecules, precursors thereof, cleavage products thereof, and combinations thereof. In addition, it is appreciated that the label on any recognition element is optionally photo bleachable, photo quenchable, or inactivatable. A recognition element is optionally bound into a single strand of growing DNA in the formation of a structure, and prior to, during, or subsequent to the addition of this recognition element the label is photo bleached such that contamination of the fluorescence of the label does not interfere with subsequent identification steps.

Identifying the presence or absence of free recognition elements in a chamber is dependent on the type of label present on the individual recognition elements. Numerous identifying methods are known in the art illustratively including far field microscopy, near field microscopy, evanescent wave or wave guided illumination, nanostructure enhancement, mass spectroscopy, photon excitation, multi photon excitation, FRET, photo conversion, spectral wavelength discrimination, fluorophore identification, background suppression, electrophoresis, surface plasma in resonance, enzyme reaction, fluorescence lifetime determination, radio frequency modulation, pulsed mutiline excitation, or combinations thereof.

It is appreciated that the structures are optionally complementary to a template structure that guides which element is placed in the next location in the sequence. Illustratively, the template structure is a DNA oligonucleotide sequence. Template DNA sequences are optionally free in solution or bound to a support in a reaction chamber or to the reaction chamber wall itself. Mobilization of the template is accomplished through conventional techniques known in the art illustratively including covalent attachment to a functional group on the solid surface, or by biotin/avidin interaction. In an optional embodiment a short oligonucleotide primer is bound to a support. The oligonucleotide segment is complementary to a small known sequence on the DNA template strand. Hybridization of the DNA template strand with the surface bound oligonucleotide immobilizes the DNA template to the surface of the chamber in reversible fashion. This embodiment has the additional advantage of providing a primer sequence for a polymerization reaction to occur. It is common in the art of DNA sequencing analyses that small segments of known sequence are present at the termination of each unknown strand. The template strand is optionally double stranded DNA, single stranded DNA, single stranded DNA hairpins, RNA, or RNA hairpins.

The inventive process further comprises a polymerization reaction in which one unknown recognition element or building element is added to the growing DNA structure in a complementary fashion. The polymerization reaction is performed by a nucleic acid polymerizing enzyme that is illustratively a DNA polymerase, RNA polymerase, reverse transcriptase, or mixtures thereof. It is further appreciated that accessory proteins or molecules are present to form the replication machinery. In a preferred embodiment the polymerizing enzyme is a thermostable polymerase or thermodegradable polymerase. Use of thermostable polymerases is well known in the art such as Taq polymerase available from Invitrogen Corporation. Thermostable polymerases allow a recognition or building reaction to be initiated or shut down by a change in temperature or other condition in the chamber without destroying activity of the polymerase.

Accuracy of the base pairing in the preferred embodiment of DNA sequencing is provided by the specificity of the enzyme. Error rates for Taq polymerase tend to be false base incorporation of 10⁻⁵ or less. Johnson, Annual Reviews of Biochemistry, 1993: 62:685-713; Kunkel, Journal of Biological Chemistry, 1992; 267:18251-18254 (both of which are hereby incorporated by reference.) Specific examples of thermostable polymerases illustratively include those isolated from Thermus aquaticus, Thermus thermophilus, Pyrococcus woesei, Pyrococcus furiosus, Thermococcus litoralis and Thermotoga maritima. Thermodegradable polymerases illustratively include E. coli DNA polymerase, the Klenow fragment of E. coli DNA polymerase, T4 DNA polymerase, T7 DNA polymerase and other examples known in the art. It is recognized in the art that other polymerizing enzymes are similarly suitable illustratively including E. coli, T7, T3, SP6 RNA polymerases and AMV, M-MLV, and HIV reverse transcriptases.

The polymerases are optionally bound to a primer template sequence. When the template sequence is a single-stranded DNA molecule the polymerase is bound at the primed end of the single-stranded nucleic acid at an origin of replication or with double stranded DNA to a nick or gap. Similarly, secondary structures such as in a DNA hairpin or an RNA hairpin allow priming to occur and replication to begin. A binding site for a suitable polymerase is optionally created by an accessory protein or by any primed single-stranded nucleic acid.

In a preferred embodiment the template is bound to a support located within the chamber. Materials suitable for forming a support optionally include glass, glass with surface modifications, silicon, metals, semiconductors, high refractive index dielectrics, crystals, gels and polymers. A support is illustratively a planar or spherical surface. It is appreciated in the inventive process that either a sequencing primer in the case of DNA sequencing, a target nucleic acid molecule, or the nucleic acid polymerizing enzyme are illustratively immobilized on the support. A complementary bonding partner for forming interactions with any of the above molecules or any other of the operational machinery in the inventive process are similarly appreciated to be suitable for immobilizing material onto a surface. Interaction of any of the replication machinery with the surface is optionally nonspecific. Examples of a specific type bonding interaction include a biotin/streptavidin linkage wherein a known primer sequence is optionally labeled with a biotin and the solid support is labeled with a streptavidin. When the biotin primer is added to the chamber a tight bonding interaction between the biotin and streptavidin occurs immobilizing the primer sequence onto the support surface. It is further appreciated that the target DNA sequence is optionally labeled itself so that it is immobilized on the support surface. Additionally, a primer sequence is optionally immobilized by hybridization with a complementary immobilized oligonucleotide. Thus a primary oligonucleotide is immobilized on a surface with a short sequence complementary to the primer oligonucleotide. It is preferred that the primer oligonucleotide is of sufficient additional length that hybridization between the immobilized nucleotide and the primer oligonucleotide allows base pairing between the primer oligonucleotide and the target DNA sequence, thus, binding the target DNA sequence to the support surface. Interaction of any suitable molecule to the support surface is appreciated to be reversible or irreversible. Alternative exemplary methods for immobilizing sequencing primer or target nucleic acid molecule to a support include antibody antigen binding pairs or photoactivated coupling molecules. It is appreciated in the art that numerous other immobilizing methods are similarly suitable in the inventive process.

It is further appreciated that the proteinaceous material of the polymerization enzyme in the case of a DNA polymerase is optionally immobilized on the surface either reversibly or irreversibly. For example, RNA polymerase was successfully immobilized on activated surface without loss of catalytic activity. Yin et al., Science, 1995; 270: 1653-57, which is hereby incorporated by reference. Alternatively, an antibody antigen pair is utilized to bind a polymerase enzyme to a support surface whereby the support surface is coated with an antibody that recognizes an epitope on the protein antigen. When the antigen is introduced into the reaction chamber it is reversibly bound to the antibody and immobilized on the support surface. A lack of interference with catalytic activity in such a method has been reported for HIV reverse transcriptase. Lennerstrand, Analytical Biochemistry, 1996; 235:141-152, which is hereby incorporated by reference. Additionally, DNA polymerase immobilization has been reported as a functional immobilization method in Korlach et al., U.S. Pat. No. 7,033,764 B2; incorporated herein by reference. Finally, any protein component can be biotinylated such that a biotin streptavidin interaction is optionally created between the support surface and the target immobilized antigen.

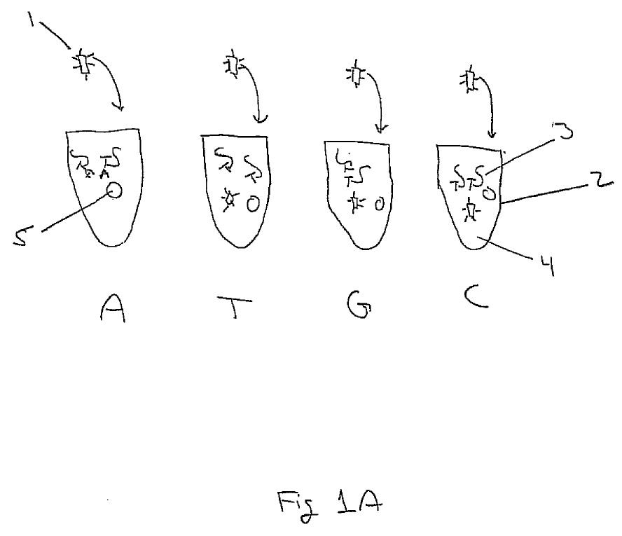

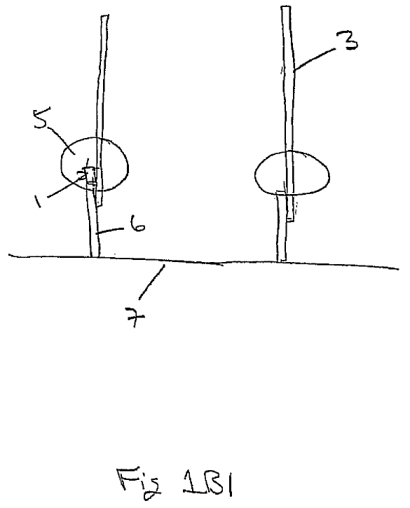

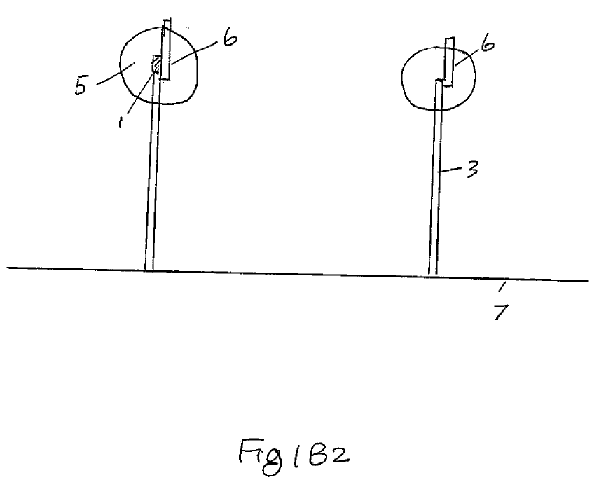

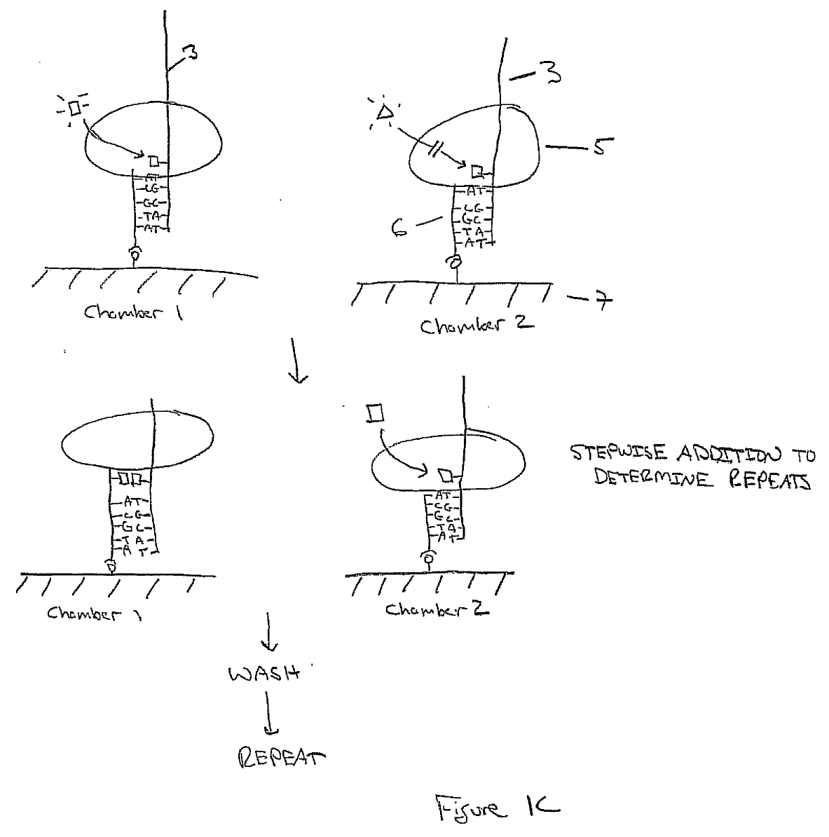

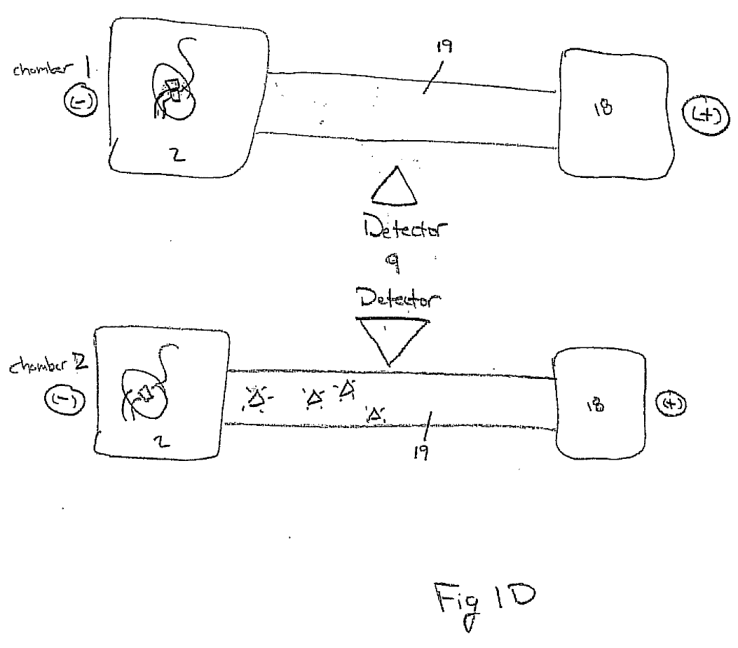

In a preferred embodiment both the target and the polymerase remain free in solution. Referring to FIG. 1, the sequencing procedure is initiated in a solution optionally containing the DNA template 3 as well as one of N species of recognition elements 1. Illustratively, four recognition element species are available represented by A, T, G and C. The four reaction chambers correspond to each species of recognition element wherein an individual species of recognition element is added. In each chamber a reaction is optionally initiated by the addition of a nucleic acid polymerizing enzyme. In an alternative embodiment a primed target sequence may be established by pre-addition of a target sequence, a primer, and a species of recognition element. No structure extension occurs in the absence of a DNA polymerase. The reaction is initiated by addition of the DNA polymerase. In an alternative embodiment all components of the replication machinery are present including the DNA polymerase, the template molecule, the primer, and a particular recognition element. The solution in this embodiment is optionally void of necessary ions for the function of the polymerase enzyme. For example, the reaction may be initiated by the addition of magnesium ions such that the replication machinery now becomes functional. In yet another alternative embodiment all of the reaction machinery is present, however, the reaction chamber is heated above a threshold temperature above the melting temperature of the template molecule and the primer such that hybridization between the primer and the template molecule does not occur. The polymerization reaction begins by adjusting the temperature to a suitable reaction temperature.

In the preferred embodiment depicted in FIG. 1A recognition element species are added to each chamber to initiate a polymerization reaction. Recognition elements are thereby diffused through the fluid medium or forced to flow through the chamber via hydrodynamic pump rapidly bringing the recognition element into association with the polymerization machinery. A recognition element inserts into the active site of the polymerase and the polymerase establishes whether this nucleotide analog is complementary to the first open base of the target nucleic acid molecule or whether a mismatch has occurred. In the reaction chamber illustrated in FIG. 1A where an A recognition element is added to the reaction chamber it is appreciated that the template at the next recognition site is defined by a T. Should an A recognition element be incorporated into the polymerase a positive match will occur and the polymerization machinery will form a covalent bond between the A and the primer sequence. However, in the second tube where a T recognition element is added a mismatch occurs and no polymerization process will proceed. If the ratio between the recognition element and the template is proper such that the recognition element illustratively is one-tenth the concentration of the template it is appreciated that all of recognition element in the A chamber will be bound and polymerized at the first hybridization site on the template molecule.

Each chamber is optionally in fluidic connection with a detector such that by washing each of the chambers free recognition elements are transported to the detector area and are readily detected. In a preferred embodiment each of the recognition elements is differentially labeled such that it can be easily distinguished from other recognition elements. Thus, a single detector is employed whereby the individual unincorporated element species are readily identified, thus, determining the sequence at the first hybridization site in the template molecule.

As depicted in FIG. 1D, an electrophoretic gel such as that formed by acrylamide, agarose, or other material known in the art is used intermediate each reaction chamber and a dedicated collection area 18 with a detector intermediate therebetween. Following sufficient time for all recognition elements to hybridize with the template strand, an electric potential is applied moving the free nucleotides past a detector 9 to the collection area. After identification of the next element in sequence, all unused elements are optionally returned to their respective recognition chambers by reversing the polarity of the electric field. This embodiment of the invention has the advantages of reducing reagent costs and time between sequencing iterations while simultaneously providing a reversible washing step for improved sequence addition.

Alternatively, numerous collection chambers are optionally employed. In a nonlimiting example each reaction chamber serves the additional roles of repeat detection chamber and sequence building chamber. Following addition of recognition elements to the recognition chambers, a first electric potential is applied to move all free recognition elements past a detector to identify the next element in sequence. This removes all unbound recognition elements from the reaction chambers. A portion or all of the reaction chambers are then used as repeat detection chambers whereby additional recognition elements are optionally added to determine the repeat number if any. A second electric potential is then applied to remove all unbound elements from the recognition chambers to a second set of collection areas. The first electric potential is then applied with reverse polarity to move all the unhybridized recognition elements back into their original recognition chambers negating the need to add more recognition elements to the N−1 chambers that did not demonstrate hybridization, thus, saving reagent and expense.

In an alternative embodiment a sampling of each of the reaction chambers or the repeat detecting chamber is obtained and injected into a mass spectrometer to recognize the presence of free elements. This embodiment has the advantage of using native, non-labeled elements whereby greater efficiency and accuracy of the polymerase is achieved. Alternatively, it is appreciated that multiple detector types are optionally employed. In a nonlimiting example, the recognition elements are fluorescently labeled. Detection of the species of hybridizing recognition element is, thus, detected by a fluorometer. After washing all unbound recognition elements from the reaction chambers, repeat detection is accomplished by addition of unlabeled recognition elements or building elements. Free elements are optionally detected by a mass spectrometer. This has the advantage of allowing washing of the chamber and use of N chambers for recognition, repeat detection, and building. Also, use of unlabeled elements in the repeat detection phase allows N replicates of repeat detection without contamination of the next round of sequence recognition.

In a preferred embodiment the template is bound to a support. Washing of the chambers removes only unbound recognition element. In an alternative embodiment the fluidic connection between each reaction chamber in the detector is such that the large template molecule remains in the chamber while the small recognition elements are readily transported through a barrier such as a size exclusion membrane or an electrophoretic gel. As such, each chamber is washed free of unbound recognition elements.

Once the complementary species of the recognition element is identified, this recognition element species is optionally stepwise added to each of the four chambers. In a nonlimiting example one-tenth concentration of recognition element was initially added to each of the four chambers for identification purposes. Stepwise addition of one-tenth concentration recognition elements allows for gradual saturation of all hybridization sites on each of the template strands. Should the stepwise additions exceed a value of ten it is understood that there is a repeat. For example if 20 stepwise additions are required before saturation of all hybridization sites on the template molecule occurs, it is appreciated that there is a single repeat on the template strand.

After identification of the particular recognition element species bound to the template molecule and determination of whether or not a repeat of that particular recognition element species occurs, building elements are added to each of the chambers at a known concentration to fully saturate all structures. At this point all unbound recognition element and building element species are optionally washed from each chamber and the reaction cycle begins again so as to determine the next recognition element species in each of the template strands. Thus, by repeating the sequence of steps the sequencing primer is extended and the entire sequence of the target is determined.

It is appreciated that the solution be of suitable extension medium so as to permit diffusion, incorporation, and washing out of each of the reaction chambers. In a nonlimiting example suitable extension media a contains 50 mM Tris-HCl pH 8.0, 25 mM magnesium chloride, 65 mM sodium chloride, 3 mM DTT, and elements at appropriate concentration to permit identification of the sequence. It is appreciated that other extension medium are similarly suitable and optimized for the particular polymerase or template being utilized.

In an alternative embodiment as in the present nonlimiting illustration, a fifth chamber is present termed a repeat detecting chamber wherein, following identification of the recognition element species, recognition element species is added to the repeat detecting chamber to determine whether or not a repeat exists and the number of repeats in sequence. Following identification of both the recognition element species in the original reaction chambers as well as the number of repeats in the repeat detecting chamber, a suitable concentration of building elements is added to all chambers to fully saturate all sites at that portion in the growing structure. It is appreciated that a washing out procedure is optionally employed between each subsequent sequence whereby the unbound elements in all reaction chambers are removed.

In an alternative embodiment the repeat detecting chamber is in fluidic communication with each of the four reaction chambers. All the reaction solution from each of the four reaction chambers is transferred to the repeat detecting chamber. It is in the repeat detecting chamber that stepwise addition of the identified species of recognition element is added to determine whether or not a repeat exists. Once the presence of a repeat is determined, or shown not to exist, the repeat detecting chamber is optionally washed free of all unbound recognition elements and the fully hybridized growing DNA molecule is subsequently transferred back to each of the four reaction chambers for the next round of element recognition.

In an alternative embodiment there is no washing out of the elements which are left in solution as excessive free elements after each of the previous steps. However, it is appreciated that the ratio between recognition elements and template is such that there is little to no observable contamination as the procedure moves through several rounds of recognition. For example, in a situation with five chambers, four recognition chambers and a repeat detecting chamber, four contain copies of free DNA molecules to be sequenced. Each chamber is initially supplied only with a small dose of one species of recognition element. This small dose is illustratively one-tenth concentration of target DNA molecules to be sequenced. After identification of the next hybridizing recognition element that element is added to the fifth chamber only using small doses to determine if there is a repeat. After the correct dose of that element is determined, the appropriate concentration of building element is added to the first four chambers and the next round of recognition begins.

In yet another alternative embodiment a sixth chamber is present termed a sequence construction chamber. Four recognition chambers contain copies of free DNA molecules to be sequenced, and each chamber is supplied with only a single species of recognition element. After the next hybridizing element is identified as described in the previous procedures, that element is added to the fifth repeat detecting chamber to determine if there is a repeat. Subsequently, the solution from all five chambers is then moved to a sixth chamber where the appropriate number of building elements for all six chambers is added to the resulting solution so as to fully saturate all free sites in the growing structure that are complementary to the current hybridization site on the template. Following saturation of all sites the solution from the sequence construction chamber transferred back to each of the four recognition chambers and the repeat detecting chamber. All chambers now contain a DNA template molecule hybridized to a growing structure of equal length and a new round of recognition element species identification occurs.

In an alternative embodiment, after identification of the next complementary recognition element each of the four recognition chambers is emptied and washed such that the solution from each of the four chambers is fully discarded and not sent to the fifth or sixth chamber. The correct amount of the determined element is added to the fifth chamber, which is a large chamber, so as to fully saturate all hybridization sites on the template DNA molecule in this chamber. Small volumes of the fifth repeat detection chamber are subsequently added back to each of the recognition chambers for a new round of recognition species identification. Optionally, a washing out procedure occurs during transfer of solution from each of the recognition chambers to a fifth repeat detecting chamber.

In an alternative process employing six chambers, five small chambers and one large sequence construction chamber, five small volumes of target DNA molecules in solution, or immobilized on a support in suspension, are transferred from the sequence construction chamber to each of the five other chambers. The next element in sequence is determined as described above. The fifth chamber is then used to determine the number of possible repeats. After the recognition element species is identified and the number of repeats is determined, the solution from all five chambers is transferred back to the sixth chamber where a correct amount of the determined element is added and the whole procedure is then repeated. It is appreciated that in this embodiment simultaneous sequencing and amplification of target DNA occurs. For example, in the situation where one large sixth sequence construction chamber is utilized small samples are withdrawn and divided amongst the four recognition chambers and the fifth repeat detecting chamber. The next element in series is identified and the presence of repeats is determined. The proper dose of building elements is then added to the sequence construction chamber to fully saturate all sites on the growing DNA structure.

It is appreciated that numerous other embodiments of the instant invention exist with greater or fewer chamber numbers, types, sizes, interconnections, or pathways and are also the subject of the instant invention.

An embodiment of the instant invention includes an apparatus. This apparatus optionally employs numerous reactor types illustratively including a batch reactor, a plug flow reactor, or a drop reactor. An apparatus for self-assembly of a number of elements comprises a reaction area that contains a suitable number of chambers relative to the number of different species of elements in the growing structure; a preparation area in fluidic connection with the reaction area whereby reagents and solutions are prepared to be delivered to the reaction area in stepwise or simultaneous fashion; and a detection area in fluidic, physical, or optical connection with the reaction area.

The detection area employs any suitable detector for detection of the type of label on each of the individual recognition elements. For example, if each of the recognition elements is labeled with a particular fluorophore a fluorescent detector is employed so as to identify which chambers contain free recognition elements. In the case where either unlabeled recognition elements are employed or nonoptically resolvable recognition elements are employed each of the reaction chambers is optionally connected to a mass spectrometer whereby the presence of free recognition elements is readily determined.

In the inventive apparatus the reaction area has N recognition chambers, each chamber having a plurality of microdispensers. The number of microdispensers is related to the number of possible recognition element species. For example, if there are four recognition element species each chamber in the reaction area has four microdispensers to allow distribution of the various species of recognition element. In an alternative embodiment there are eight microdispensers aimed at each of the reaction chambers such that any of the four recognition elements are optionally distributed to each reaction chamber as well as any building elements without fear of contamination between the elements. Thus, each microdispenser is filled with one type of element so that each type of element is available to be distributed into each chamber in the reaction area. In the case of five small chambers the fifth chamber similarly has four or eight microdispensers for delivery of elements to that chamber. It is appreciated that the number of microdispensers is optionally related to the number of the elements in the growing structure. In the case of ten separate element species as many as ten or twenty microdispensers for each chamber are employed. Alternatively, a single or fewer than N microdispensers is employed with a washing out step of each of the microdispensers between delivery of different recognition or building elements.

It is appreciated that the reaction area contains no moving parts. Fluidic connection between each of the chambers is optionally powered by differential electric potential so as to move free recognition or building elements between the chambers. Further, DNA template and growing structure may similarly be transferred between chambers.

### EXAMPLE 1

A standard reaction chamber protocol is outlined in FIG. 1C. DNA template with a known termination sequence of 3′-CAT TTT GCT GCC GGT CA- . . . -5′ (SEQ ID No. 1) is amplified by standard PCR techniques and purified on an anion exchange resin supplied by Quiagen, Inc., Valencia, Calif. 400 ng of template is added to each of four reaction chambers, a repeat detection chamber, and a sequence building chamber each containing a reaction solution of 60 mM Tris-SO₄ (pH 8.9), 180 mM Ammonium Sulfate. A primer (8 μg) of complementary sequence 5′-GTA AAA CGA CGG CCA GT-3′ (SEQ ID No. 2) is added to each chamber and allowed to hybridize with the DNA template under suitable conditions. A single species of Fluorecein-12 labeled A, T, G, and C nucleotides obtained from Perkin Elmer, Waltham, Mass. are added to each of the four reaction chambers 1/10 mol/mol concentration relative to DNA template. A polymerization reaction is initiated by the addition of 1 unit (final) of Platinum® Taq DNA Polymerase (Invitrogen, Inc., Carlsbad, Calif.) along with 2 mM MgSO₄ (final) in reaction solution. The reaction is allowed to proceed for 5 sec. An electric potential is applied to the solution of each reaction chamber in sequence whereby free nucleotide is selectively moved from the reaction chamber toward a detection area in which a fluorescent detector determines whether a fluorescent nucleotide is present in solution. Fluorescent parameters are 496 nm excitation, 517 nm emission with a 5 nm bandpass filter. Identification of which reaction chamber does not possess free labeled recognition element determines which element is next in sequence. The reaction chamber in which a labeled A was added demonstrates no free nucleotide.

Fluorescein-12 labeled A is added to the repeat detection chamber along with DNA polymerase, MgSO₄, and reaction solution by a microdispenser in 1/10 mol/mol amounts in sequential fashion and the reaction is allowed to proceed for 5 sec followed by application of an electric potential to determine if free nucleotide is present in solution. It is appreciated that other relative amounts of nucleotide and template are similarly suitable in all chambers. Application of an electric potential moves free nucleotide to a detector area where the presence of free nucleotide is determined as above. The process in the repeat detection chamber is repeated until free nucleotide recognized by the detector. Twenty additions are required for the instant exemplary template strand indicating that there is an AA repeat sequence.

2×mol/mol concentration of unlabeled A nucleotide (building element) is added to each of the reaction chambers and the sequence building chamber and the polymerization is allowed to proceed for 2 min followed by application of an electric potential to wash out any remaining free recognition or building element from all chambers.

The process is repeated for 350 cycles to fully assemble and identify the sequence of all nucleotide elements in the DNA sequence.

### EXAMPLE 2

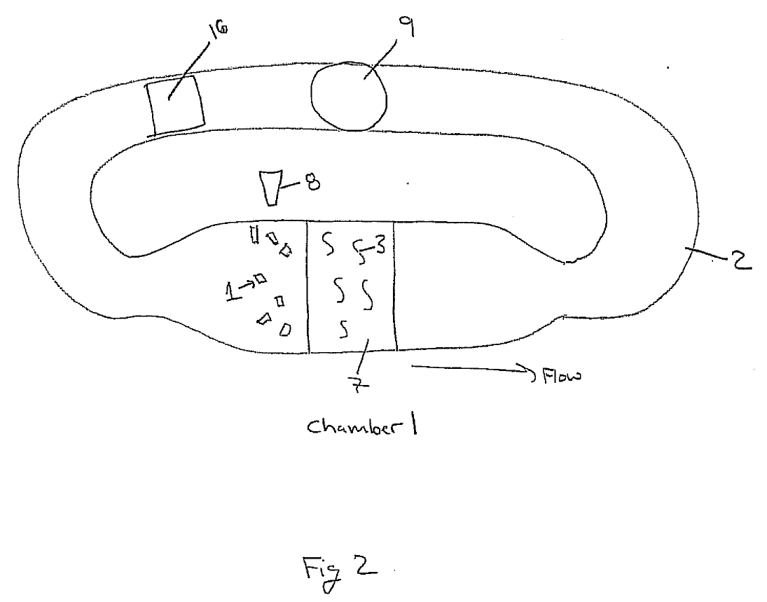

Referring to FIG. 2, a reaction chamber 2 is depicted as illustrated by a tubular loop structure wherein a support 7 is coated on a portion thereof which contains reaction solution. The support is coated with streptavidin by techniques known in the art. The primer of Example 1 is biotinylated by techniques known in the art illustratively by incorporation of biotin-aha-CTP (Invitrogen) in the primer sequence at the 5′ end. The primer is added to the reaction chamber and allowed to interact with the support. DNA template is then added at a concentration such that nearly all the DNA template will be hybridized with primer. Recognition elements 1, polymerase 5, and initiation ions such as in Example 1 are added by a microdispenser to the reaction chamber and a pump 16 circulates the fluid in the chamber such that the recognition element is flowed across the template bound support for 10 seconds with continuous monitoring by the fluorescent detector 9. Over the course of the reaction time the reaction chamber that contains the complementary recognition element demonstrates a reduction in fluorescence indicating that the element is incorporated onto the support bound primer. Analyses of each respective reaction chamber identify the next nucleotide in sequence. Two other similar reaction chambers, or standard container chambers are employed for the repeat detection chamber and the sequence building chamber and the structure building reaction and sequence identification is completed by subsequent iterative steps.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A process of self-assembling a number of elements into a structure comprising:
providing N recognition chambers,
adding a plurality of templates into a solution present in each chamber of said N recognition chambers;
introducing a unique plurality of a homogeneous species of recognition elements to each chamber of said N recognition chambers;
exposing said plurality of templates and said unique plurality of recognition elements in each of said N recognition chambers to a polymerization reaction with a plurality of polymerization enzymes in each of said N recognition chambers;
identifying a next in sequence recognition element on said plurality of templates in at least one of said N recognition chambers;
placing building elements corresponding to said next in sequence recognition element in at least one of said N recognition chambers;
subjecting said plurality of templates and said building elements to said polymerization reaction in at least one of said N recognition chambers;
repeating the introducing through subjecting steps until said structure is complete.

2. The process of claim 1, further comprising determining said template sequence.

3. The process of claim 1, her comprising washing all elements from N chambers prior to adding building elements.

4. The process of claim 1, wherein N is the number of unique recognition elements.

5. The process of claim 1, where N is 4.

6. The process of claim 1, wherein said recognition elements are selected from the group comprising: nucleotides, ribonucleotides, deoxynucleotides, dideoxynucleotides, peptide nucleotides, modified nucleotides, modified peptide nucleotides, modified phosphate-sugar backbone nucleotides, amino acids, and modified amino acids.

7. The process of claim 1, wherein building elements are selected from the group comprising: nucleotides, ribonucleotides, deoxynucleotides, dideoxynucleotides, peptide nucleotides, modified nucleotides, modified peptide nucleotides, modified phosphate-sugar backbone nucleotides, amino acids, and modified amino acids.

8. The process of claim 1, further comprising:
placing in a repeat detecting chamber at least said plurality of templates and the solution wherein detecting repeat elements occurs by stepwise addition of building elements or recognition elements corresponding to said next in sequence recognition element; and
calculating the number of repeat elements in the sequence of said template.

9. The process of claim 8, further comprising transferring said solution from all N recognition chambers to said repeat detecting chamber prior to adding said building elements or recognition elements.

10. The process of claim 9, further comprising:
transferring said solution from said repeat detecting chamber back to said N recognition chambers prior to said adding said unique plurality of recognition elements.

11. The process of claim 8, further comprising:
transferring said solution from said N recognition chambers and said solution from said repeat detection chamber to a sequence construction chamber;
adding building elements corresponding to said next in sequence recognition element to said at least one of said N recognition chambers.

12. The process of claim 11, further comprising transferring said solution from said sequence construction chamber to all of said N recognition chambers and to said repeat detection chamber prior to adding said unique plurality of recognition elements.

13. The process of claim 1, wherein said plurality of templates is immobilized.

14. The process of claim 1, wherein said plurality of templates is in solution.

15. The process of claim 1, further comprising washing out said unique plurality of recognition elements or said building elements after exposure to conditions of said polymerization reaction.

16. The process of claim 1, wherein said polymerization enzyme is a family of polymerization catalyzing molecules.

17. The process of claim 1, wherein said polymerization enzyme is attached to a support.

18. The process of claim 1, wherein said chamber is in a reactor selected from the group comprising a batch reactor, a plug flow reactor, and a drop reactor.

19. The process of claim 1, wherein said polymerization reaction is performed by a nucleic acid polymerization enzyme selected from the group comprising a DNA polymerase, an RNA polymerase, reverse transcriptase, and mixtures thereof.

20. The process of claim 19, wherein said nucleic acid polymerizing enzyme is a thermostable polymerase or thermodegradable polymerase.

21. The process of claim 1, wherein the template is selected from the group comprising double-stranded DNA, single-stranded DNA, single-stranded DNA hairpins, DNA, and RNA hairpins.

22. The process of claim 1, wherein said solution further comprises a primer sequence.

23. The process of claim 1, wherein said unique plurality of recognition elements further comprise a label or a plurality of labels.

24. The process of claim 23, wherein said label is selected from the group comprising chromophores, fluorescent moieties, haptens, enzymes, antigens, dyes, phosphorescent groups, chemiluminescent moieties, scattering or fluorescent nanoparticles, Ramen signal generating moiety, photobleachable label, photoquenchable label, activatable or inactivatable label, precursors of any of the previous, cleavage products of any of the previous, and combinations of any of the previous, or a nullity.

25. The process of claim 23, wherein said label is attached to said recognition elements at a base, sugar moiety, alpha-phosphate, beta-phosphate; gamma-phosphate, or combinations thereof.

26. The process of claim 23, wherein each homogeneous species of recognition elements has a distinguishable label or plurality of labels.

27. The process of claim 23, wherein three or fewer homogeneous species of said unique plurality of recognition elements has a distinguishable label or plurality of labels.

28. The process of claim 1, wherein said identifying is performed by procedures selected from the group comprising far field microscopy, near field microscopy, evanescent wave or wave guided illumination, photon excitation, multiphoton excitation, FRET, photoconversion, spectral wavelength discrimination, fluorophore identification, background suppression, nanostructure enhancement, mass spectroscopy, chromatography, electrophoresis, surface plasmon resonance, enzyme reaction, fluorescence lifetime, radio frequency modulation, pulsed multiline excitation, or combinations thereof.

29. The process of claim 23, further comprising photobleaching, cleaning, or inactivating said label, or cleaving said label from said recognition element.

30. The process of claim 1, wherein each template is hybridized to a separate oligonucleotide.

31. The process of claim 30, wherein said separate oligonucleotide is attached to a support.

32. The process of claim 23, wherein said identifying is achieved by detecting at least one of said label.

33. An apparatus for self-assembly of a number of elements comprising:
a reaction area;
a preparation area in fluidic connection with said reaction area;
a detection area in fluidic, physical, or optical connection with said reaction area;
said reaction area having no moving parts;
said reaction area having N recognition chambers;
each chamber having a plurality of microdispensors, each of said microdispensors capable of dispensing a unique species of recognition element or building element.

34. The apparatus of claim 33, where N is 4.

35. The apparatus of claim 33, wherein said reactions area is a batch flow reactor, a plug flow reactor, or a drop reactor.

36. The apparatus of claim 33, further comprising a repeat detecting chamber.

37. The apparatus of claim 36 wherein said repeat detecting chamber is in fluidic connection with said recognition chambers.

38. The apparatus of claim 33, further comprising a sequence construction chamber.

39. The apparatus of claim 38, wherein said sequence construction chamber is in fluidic connection with said recognition chambers and said repeat detecting chamber.
